(19) United States
(12) Patent Application Publication
(10) Pub. No.: US 2009/0005576 A1
(43) Pub. Date: Jan. 1, 2009

(54) Production Method of Optically Active Cyclohexane Ether Compounds

(75) Inventors: Koji Machiya, Tokyo (JP); Kazuo Ike, Tokyo (JP); Masaru Watanabe, Tokyo (JP); Toshitaka Yoshino, Tokyo (JP); Takumi Okamoto, Tokyo (JP); Yasuhiro Morinaga, Tokyo (JP); Shoji Mizobata, Tokyo (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Astellas Pharma Inc., Tokyo (JP)

(21) Appl. No.: 11/813,809

(22) PCT Filed: Jan. 12, 2006
(86) PCT No.: PCT/JP2006/300667

§ 371 Date: Jul. 12, 2007

Related U.S. Application Data
(60) Provisional application No. 60/642,998, filed on Jan. 12, 2005.

(51) Int. Cl. C07D 207/12

(52) U.S. Cl. 548/541

(57) ABSTRACT

The present invention relates to an industrial synthetic method of an optically active cyclohexane ether compound (IIIa) or a salt thereof, which is useful as a pharmaceutical agent, and an intermediate useful for the production method of the present invention. The production method of the present invention is as shown below: wherein each symbol is as defined in the specification. According to the production method of the present invention, efficient and stable supply of an optically active cyclohexane ether compound (IIIa) in a high yield at a lower cost can be afforded. Therefore, an optically active cyclohexane ether compound (IIIa) extremely useful as a pharmaceutical agent can be provided by an industrially highly advantageous method.

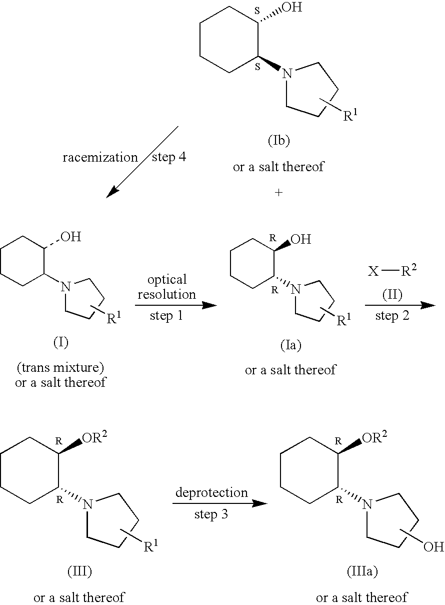

### TECHNICAL FIELD

The present invention relates to an industrial production method of an optically active cyclohexane ether compound.

### BACKGROUND ART

A compound represented by the formula (IIIa) in the specification is described in WO2004/099137 and WO2004/098525, has an ion channel controlling action and is useful as a prophylaxis and/or therapeutic agent for various diseases such as arrhythmia (atrial arrhythmia, ventricular arrhythmia), fibrillation (atrial fibrillation, ventricular fibrillation), flutter (atrial flutter, ventricular flutter) and so on. In WO2004/099137, for example, compound A is produced from an easily available starting material according to the route shown below in Scheme 1.

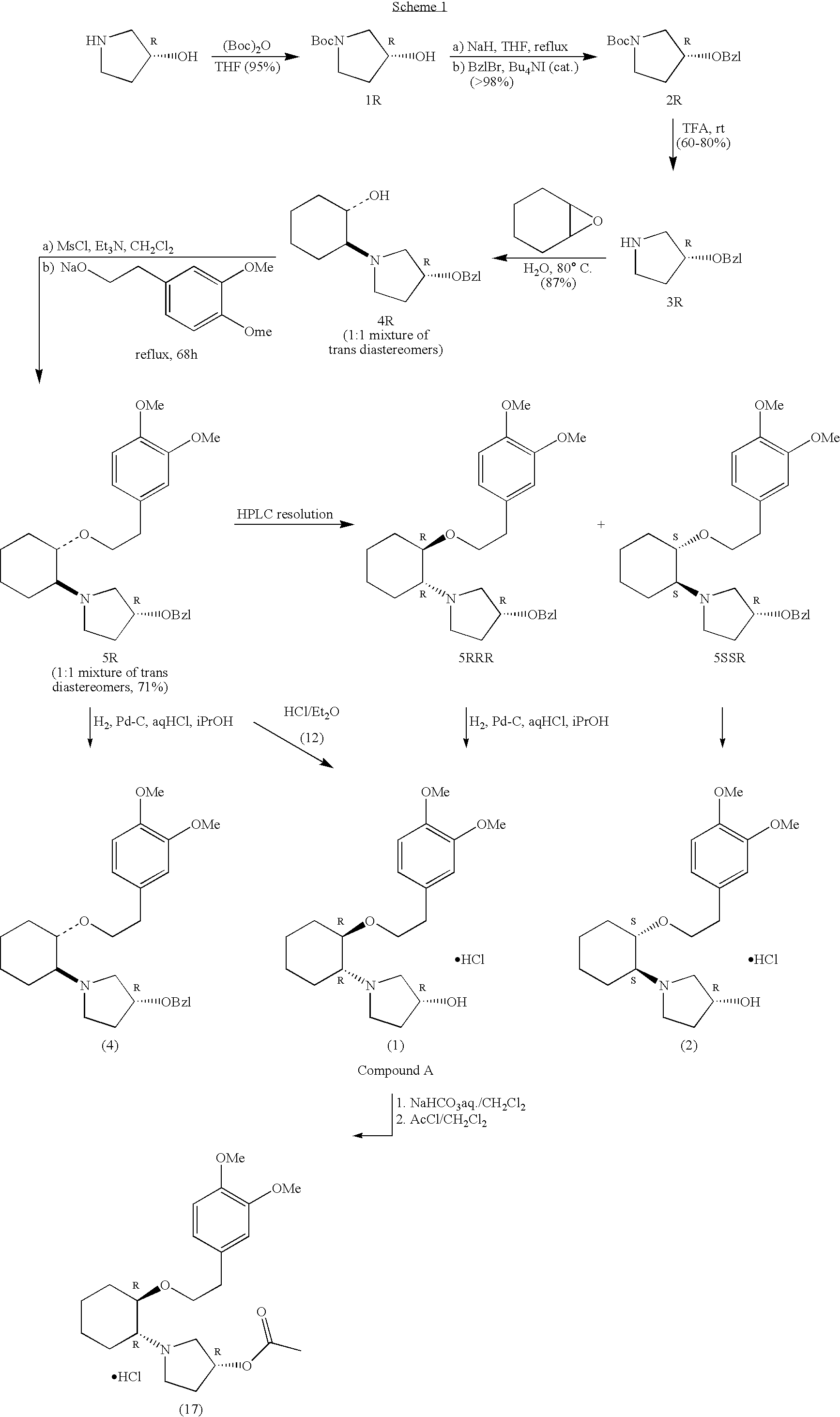

In the above-mentioned route, compound (5R) is produced by reacting compound (4R) with methanesulfonyl chloride to convert OH group to chloro group and reacting the resulting compound with 3,4-dimethoxyphenethyl alcohol (DMPE) in the presence of sodium hydride. This compound (5R) is a trans mixture due to two asymmetric carbon atoms on the cyclohexane ring and includes two kinds of diastereomers (5RRR and 5SSR) (ca. 1:1). Thus, optical resolution is performed in the next step to give a single optically active form (5RRR), which is subjected to debenzylation to give compound A. However, since this optical resolution is performed by HPLC, a solvent is necessary in large amounts, and separation efficiency is poor because the processing takes time etc., thus posing many problems in terms of cost and supply as an industrial production method. Further, satisfactory results could not be achieved by crystallization in optical resolution of the compound (5R) when the present inventors studied the optical resolution. In addition, since DMPE is expensive, optical resolution after reaction with DMPE problematically leads to high cost. Furthermore, about half of the compound (5R) obtained through 5 steps from the starting material cannot be utilized for the synthesis of compound A, which in turn increases the cost. Related to trans-2-cyclicaminocyclohexanol compounds, two prior arts are found. In J. Medicinal Chemistry, 1217, 32 (1989), it is described that di-p-toluoyl-L-tartaric acid as an optical resolution agent is used for trans-2-(4-phenylpiperidino)cyclohexanol in fractional crystallization by salt formation. Trans-2-(4-phenylpiperidino)cyclohexanol has two asymmetric carbon atoms on cyclohexane ring, but the substituent of the cyclohexane ring is piperidino rather than pyrrolidinyl. In Tetrahedron Asymmetry, 10, 2307-2310 (1999), it is described that trans-(±)-2-(pyrrolidinyl)cyclohexanol was optically resolved using chiral 1,1′-bi-2-naphthol and boric acid as optical resolution agents, on the other hand, it is described that optical resolution of the compound using tartaric acid and binaphthylphosphoric acid did not obtain successful results. Trans-(±)-2-(pyrrolidinyl)cyclohexanol also has two asymmetric carbon atoms.

### DISCLOSURE OF THE INVENTION

The present invention has been made as a result of intensive studies of a production method of a compound represented by the formula (IIIa) including compound A useful as a pharmaceutical agent, which enables more efficient and stable supply in a high yield at a lower cost. The present inventors have found that more efficient and stable supply of a compound represented by the formula (IIIa) in a high yield at a lower cost is enabled by performing optical resolution without using HPLC (preferably by crystallization, more preferably by fractional crystallization by salt formation) and performing optical resolution before reaction with a reagent such as DMPE, which in turn enables production of a compound represented by the formula (IIIa) including compound A by an industrially highly advantageous method. Moreover, the present inventors have found that, by racemization, an other optically active compound, which is a diastereomer of the object optically active compound obtained by optical resolution is converted to a trans mixture which is a compound before optical resolution, and the resulting trans mixture can be subjected to the optical resolution again, whereby a compound represented by the formula (IIIa) including compound A can be produced by a method capable of reducing the cost further.

Accordingly, the present invention provides the following.
(1) A production method of an optically active compound represented by the formula (IIIa):

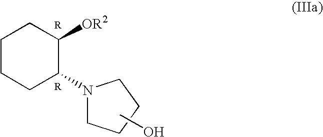

wherein R² is an optionally substituted aryl lower alkyl group (hereinafter to be also referred to as compound (IIIa)), or a salt thereof, which comprises subjecting a compound represented by the formula (I), which is a trans mixture:

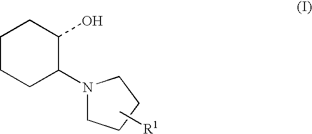

wherein R¹ is an optionally protected hydroxy group (hereinafter to be also referred to as compound (I)), or a salt thereof, to optical resolution (preferably by crystallization, more preferably by fractional crystallization by salt formation) to give an optically active compound represented by the formula (Ia):

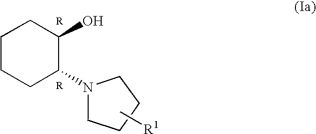

wherein R¹ is as defined above (hereinafter to be also referred to as compound (Ia)), or a salt thereof, reacting the resulting compound or a salt thereof with a compound represented by the formula (II): X—R² wherein R² is as defined above and X is a leaving group (hereinafter to be also referred to as compound (II)), to give an optically active compound represented by the formula (III):

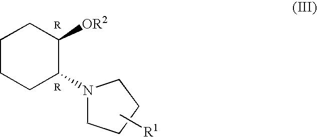

wherein R¹ and R² are as defined above (hereinafter to be also referred to as compound (III)), or a salt thereof, and then eliminating, when R¹ is a protected hydroxy group, the hydroxyl-protecting group from the resulting compound or a salt thereof.
(2) A production method of an optically active compound represented by the formula (III):

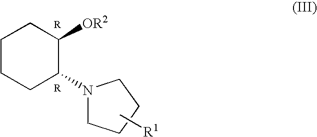

wherein R¹ is an optionally protected hydroxy group, and R² is an optionally substituted aryl lower alkyl group, or a salt thereof, which comprises subjecting a compound represented by the formula (I), which is a trans mixture:

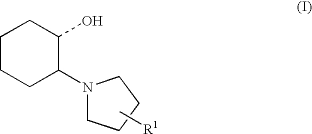

wherein R¹ is as defined above, or a salt thereof, to optical resolution (preferably by crystallization, more preferably by fractional crystallization by salt formation) to give an optically active compound represented by the formula (Ia):

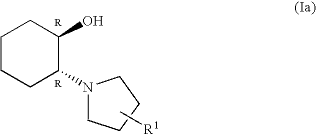

wherein R¹ is as defined above, or a salt thereof, and then reacting the resulting compound or a salt thereof with a compound represented by the formula (II): X—R² wherein R² is as defined above and X is a leaving group.
(3) A production method of an optically active compound represented by the formula (Ia):

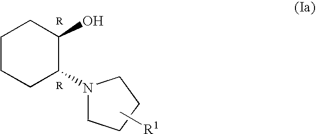

wherein R¹ is an optionally protected hydroxy group, or a salt thereof, which comprises subjecting a compound represented by the formula (I), which is a trans mixture:

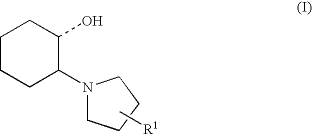

wherein R¹ is as defined above, or a salt thereof, to optical resolution (preferably by crystallization, more preferably by fractional crystallization by salt formation).
(4) A production method of an optically active compound represented by the formula (IIIa):

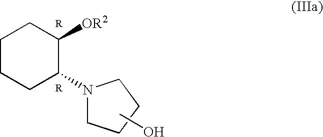

wherein R² is an optionally substituted aryl lower alkyl group, or a salt thereof, which comprises reacting an optically active compound represented by the formula (Ia):

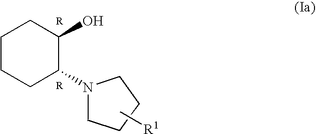

wherein R¹ is an optionally protected hydroxy group, or a salt thereof, with a compound represented by the formula (II): X—R² wherein R² is as defined above and X is a leaving group, to give an optically active compound represented by the formula (III):

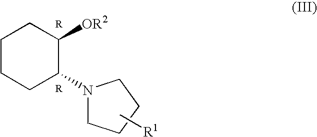

wherein R¹ and R² are as defined above, or a salt thereof, and then eliminating, when R¹ is a protected hydroxy group, the hydroxyl-protecting group from the resulting compound or a salt thereof.
(5) A production method of an optically active compound represented by the formula (III):

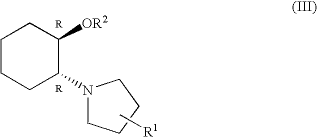

wherein R¹ is an optionally protected hydroxy group, and R² is an optionally substituted aryl lower alkyl group, or a salt thereof, which comprises reacting an optically active compound represented by the formula (Ia):

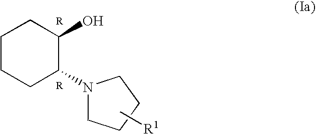

wherein R¹ is as defined above, or a salt thereof, with a compound represented by the formula (II): X—R² wherein R² is as defined above and X is a leaving group.
(6) A production method of a compound represented by the formula (I), which is a trans mixture:

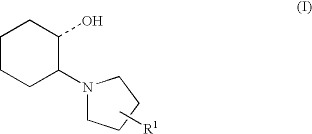

wherein R¹ is an optionally protected hydroxy group, or a salt thereof, which comprises subjecting an optically active compound represented by the formula (Ib):

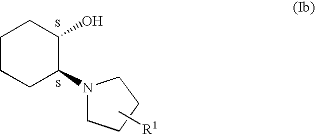

wherein R¹ is as defined above, (hereinafter to be also referred to as compound (Ib)), or a salt thereof, to a racemization.
(7) The production method of any of the above-mentioned (1) to (3), wherein the compound represented by the formula (I), which is a trans mixture:

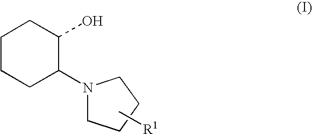

wherein R¹ is as defined above, or a salt thereof, is obtained by the production method of the above-mentioned (6).
(8) An optically active compound represented by the formula (Ia):

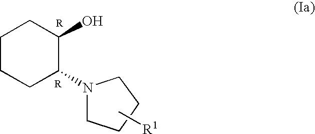

wherein R¹ is an optionally protected hydroxy group, or a salt thereof.

### BRIEF DESCRIPTION OF THE DRAWINGS

FIG. 1 is a HPLC chart when the presence of the aziridinium salt (the peak of 9.71 min) and 1-chloro compound (the peak of 12.79 min) was confirmed by HPLC in the racemization step of Example 4.

FIG. 2 is a HPLC chart when the disappearance of the aziridinium salt and 1-chloro compound was confirmed by HPLC in the racemization step of Example 4.

The peak of 10.17 min represented the presence of compound (1).

### BEST MODE FOR EMBODYING THE INVENTION

The present invention is explained in detail in the following. In the present invention, each group is defined as follows.

The term “lower” means having 1 to 6 carbon atoms, unless otherwise specified.

As the hydroxyl-protecting group of the “optionally protected hydroxy group” for R¹, a suitable one can be found in T. W. Greene and P. G. M. Wuts, “Protective Groups in Organic Synthesis” (John Wiley & Sons, Inc., 3rd Edition), the content of which is incorporated herein by reference, such as phenyl(lower)alkyl optionally having one or more suitable substituent(s), (e.g., benzyl, 4-methoxybenzyl, trityl etc.), phenylcarbonyl optionally having one or more suitable substituent(s), (e.g., benzoyl, 4-methoxybenzoyl etc.), tri-substituted silyl [for example, lower alkylsilyl (e.g., trimethylsilyl, tert-butyldimethylsilyl etc.) and the like], tetrahydropyranyl and the like.

As the “aryl” of the “optionally substituted aryl lower alkyl group” for R², C₆₋₁₀ aryl such as phenyl, naphthyl, pentalenyl and the like can be mentioned, of which phenyl is particularly preferable.

The “lower alkyl group” of the “optionally substituted aryl lower alkyl group” for R² may be a linear or branched chain. As preferable specific examples, methyl, ethyl, 1-propyl, isopropyl, 1-butyl, isobutyl, tert-butyl, sec-butyl, 1-pentyl, isopentyl, sec-pentyl, tert-pentyl, methylbutyl, 1,1-dimethylpropyl, 1-hexyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 2,2-dimethylbutyl, 3,3-dimethylbutyl, 1-ethyl-1-methylpropyl and the like can be mentioned.

The “aryl lower alkyl group” of the “optionally substituted aryl lower alkyl group” for R² is optionally substituted by one or more substituent(s), and as the substituent, lower alkyl group, lower alkenyl group, lower alkynyl group, cyclo(lower)alkyl group, cyclo(lower)alkenyl group, cyclo(lower)alkyl lower alkyl group, aryl group, aryl lower alkyl group, halo(lower)alkyl group, lower alkoxy group, aryloxy group, hydroxy group, protected hydroxy group (preferable protected hydroxy group are described in the aforementioned T. W. Greene and P. G. M. Wuts, “Protective Groups in Organic Synthesis”), mono- or di-lower alkylamino group, lower alkyl-carbonyl group, lower alkoxy-carbonyl group and the like can be mentioned. Of these, “lower alkoxy group” is preferable. As specific examples, methoxy, ethoxy, propoxy, isopropoxy, butoxy, sec-butoxy, tert-butoxy, pentyloxy, hexyloxy and the like can be mentioned, and methoxy is particularly preferable.

R² is preferably aryl lower alkyl group substituted by two lower alkoxy groups, and phenyl lower alkyl group substituted by two lower alkoxy groups is more preferable, and dimethoxyphenylethyl group is particularly preferable, and 3-4-dimethoxyphenylethyl group is most preferable.

As the “leaving group” for X, conventionally known leaving groups can be mentioned, such as halogen atom (fluorine atom, chlorine atom, bromine atom, iodine atom), methanesulfonyloxy group, p-toluenesulfonyloxy group and the like, and

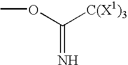

wherein X¹ is a halogen atom, and the like can be mentioned. Of these,

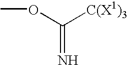

is preferable, and

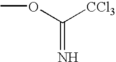

is particularly preferable.

As compound (I), which is a starting material in the production method of the present invention,

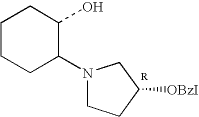

is preferable, and therefore, as the objective compound (IIIa), compound A, i.e.,

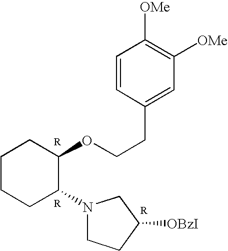

is preferable.

The production method of the present invention is now explained in the following.

The production method of the present invention comprises the following route.

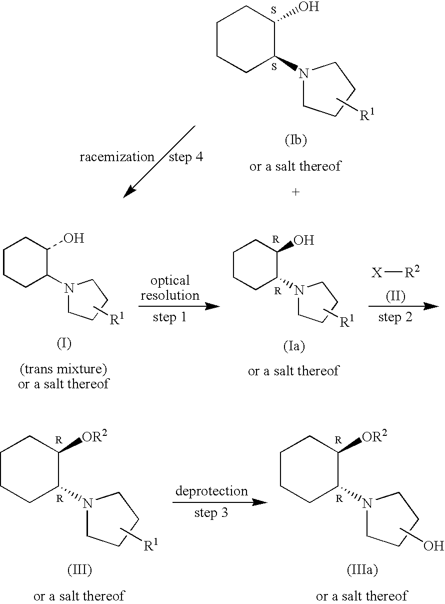

wherein each symbol is as defined above.

### Step 1 (Optical Resolution)

In this step, optically active compound (Ia) (RR form) or a salt thereof can be produced by subjecting compound (I), which is a trans mixture, or a salt thereof to optical resolution. With regard to the cyclohexane ring in compound (I), the carbon atom that the OH group is bonded to and the carbon atom that the pyrrolidinyl group is bonded to are both asymmetric carbon atoms, and the OH group and the pyrrolidinyl group are at the trans position to each other. The compound (I) is a mixture of RR form (compound (Ia)) and SS form (compound (Ib)), where the mixing ratio thereof is not particularly limited.

The method for optical resolution employed in this step preferably comprises crystallization, more preferably, comprises fractional crystallization by forming a salt with an optical resolution agent, in this instance, the resulting salt can be converted to a free form thereof by neutralization.

As the optical resolution agent to be used in this step, one suitable for compound (I) is used and the amount thereof to be used is generally 0.5 to 2 equivalents relative to 1 equivalent of compound (I) or a salt thereof.

The solvent to be used in the step is one that does not adversely influence the optical resolution, and a solvent suitable for compound (I) and an optical resolution agent can be used. For example, esters such as ethyl acetate, methyl acetate and the like; ethers such as diethyl ether, tetrahydrofuran and the like; hydrocarbons such as toluene and the like; halogenated hydrocarbons such as chloroform, dichloromethane and the like; alcohols such as methanol, ethanol, isopropyl alcohol and the like; water and the like can be mentioned. These solvents are used alone or in combination.

In addition, while the conditions (reaction temperature etc.) for optical resolution are also those suitable for compound (I) and optical resolution agent, optical resolution is generally performed under cooling or heating.

As the optical resolution agent, di-p-toluoyl-L-tartaric acid is preferably used, and as the solvent then, ethyl acetate is preferable. When these optical resolution agent and solvent are used, an RR form with high purity is preferentially crystallized as compound (Ia), and recrystallization as necessary can give the optically active compound having a high purity of about 99% or more.

The neutralization is carried out by adding, in a solvent, a base to a crystal after fractional crystallization.

As the base to be used for neutralization, inorganic bases and organic bases, for example, alkali metals (e.g., sodium, potassium etc.), alkaline earth metals (e.g., magnesium, calcium etc.), hydroxide, carbonate and bicarbonate thereof, trialkylamines (e.g., trimethylamine, triethylamine etc.) can be mentioned. Of these, inorganic bases are preferable, hydroxide, carbonate and bicarbonate of alkali metals (e.g., sodium, potassium etc.) are more preferable, and sodium hydroxide is particularly preferable.

As the solvent to be used for neutralization, a solvent suitable for a crystal after fractional crystallization can be used. For example, esters such as ethyl acetate, methyl acetate and the like; ethers such as diethyl ether, tetrahydrofuran and the like; hydrocarbons such as toluene and the like; halogenated hydrocarbons such as chloroform, dichloromethane and the like; alcohols such as methanol, ethanol, isopropyl alcohol and the like; water and the like can be mentioned. These solvents are used alone or in combination.

The compound (I) can be produced according to a method known per se, such as a method described in WO2004/099137 or a method analogous thereto.

### Step 2 (Etherification)

In this step, optically active compound (III) or a salt thereof can be produced by reacting optically active compound (Ia) or a salt thereof obtained in Step 1 with compound (II) represented by X—R².

As compound (II), a compound having a group suitable for reaction with compound (Ia) can be used, a compound (II) wherein the leaving group X is

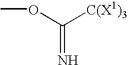

wherein X¹ is as defined above (trihaloacetimidate), preferably chlorine atom (trichloroacetimidate), is preferably used.

The amount of compound (II) to be used is generally 1-2 mol relative to 1 mol of compound (Ia) or a salt thereof.

The solvent to be used in the step is one that does not adversely influence the reaction and a solvent suitable for compound (Ia) or a salt thereof and compound (II) can be used. For example, esters such as ethyl acetate, methyl acetate and the like; ethers such as diethyl ether, tetrahydrofuran and the like; hydrocarbons such as benzene, toluene and the like; halogenated hydrocarbons such as chloroform, dichloromethane and the like; dimethyl sulfoxide and the like can be mentioned. These solvents are used alone or in combination. Of these, hydrocarbons such as benzene, toluene and the like are particularly preferable.

This step is generally performed using a catalytic amount of a Lewis acid (e.g., BF₃—OEt₂, TMSOTf (trimethylsilyl trifluoromethanesulfonate) etc.). When compound (II) is a trihaloacetimidate, fine reactivity can be afforded by the use of a strong organic acid such as trifluoromethanesulfonic acid and the like. The amount of the strong organic acid to be used is preferably 1 to 2 mol, particularly preferably 1.1 to 1.6 mol, relative to 1 mol of compound (Ia) or a salt thereof. The reaction temperature in this case is preferably 0 to 50° C., and the reaction time is preferably 0.5 to 10 hr.

In this step, the steric configuration of compound (Ia) or a salt thereof is retained as it is in the obtained compound (III) or a salt thereof. When compound (Ia) is reacted with methanesulfonyl chloride in the same manner as in conventional methods, the steric configuration cannot be retained.

The compound (II) is produced according to a method known per se, and when, for example, compound (II) is trichloroacetimidate, it can be produced according to a method described in Chem. Rev. 1993, 93, 1503-1531; Tetrahedron Letters 1996, 37, 1481-1484 or methods analogous thereto.

### Step 3 (Deprotection)

In this step, compound (IIIa) or a salt thereof can be produced by eliminating the hydroxy-protecting group from compound (III) or a salt thereof. The above-mentioned deprotection is carried out according to a conventional method such as hydrolysis, reduction and the like.

Hydrolysis is preferably carried out in the presence of base or acid such as Lewis acid and the like. As preferable base, inorganic bases and organic bases, for example, alkali metals (e.g., sodium, potassium etc.), alkaline earth metals (e.g., magnesium, calcium etc.), hydroxide, carbonate and bicarbonate thereof, trialkylamines (e.g., trimethylamine, triethylamine etc.) can be mentioned. As preferable acid, organic acids (e.g., formic acid, acetic acid, propionic acid, trichloroacetic acid, trifluoroacetic acid etc.) and inorganic acids (e.g., hydrochloric acid, hydrobromic acid, sulfuric acid, hydrogen chloride, hydrogen bromide etc.) can be mentioned.

The amount of the base or acid to be used is generally 0.5 to 100 mol relative to 1 mol of compound (III) or a salt thereof.

The solvent to be used for the hydrolysis is a solvent that does not adversely influence the reaction and a solvent suitable for compound (III) or a salt thereof can be used. For example, water, methanol, ethanol, tetrahydrofuran and the like can be mentioned. These solvents are used alone or in combination.

As the preferable reducing agent to be used for chemical reduction, metals (e.g., tin, zinc, iron etc.), and a combination of a metal compound (e.g., chromium chloride, chromium acetate etc.) and an organic acid or inorganic acid (e.g., formic acid, acetic acid, propionic acid, trifluoroacetic acid, p-toluenesulfonic acid, hydrochloric acid, hydrobromic acid etc.) can be mentioned.

As preferable catalyst to be used for the catalytic reduction, conventional catalysts such as platinum catalysts (e.g., platinum plate, platinum sponge, platinum-black, colloidal platinum, platinum oxide, platinum wire etc.), palladium catalysts (e.g., palladium sponge, palladium-black, palladium oxide, palladium-carbon, cholloidal palladium, palladium-barium sulfate, palladium-barium carbonate etc.), nickel catalysts (e.g., reduced nickel, nickel oxide, Raney-nickel etc.), cobalt catalysts (e.g., reduced cobalt, Raney-cobalt etc.), iron catalysts (e.g., reduced iron, Raney-iron etc.), copper catalysts (e.g., reduced copper, Raney-copper etc.) and the like can be mentioned.

The solvent to be used for the reduction is a solvent that does not adversely influence the reaction, and a solvent suitable for compound (III) or a salt thereof can be used. In the case of chemical reduction, for example, water, methanol, ethanol, propanol, N,N-dimethylformamide and the like can be mentioned, and in the case of catalytic reduction, the above-mentioned solvents and other conventional solvents such as diethyl ether, dioxane, tetrahydrofuran and the like can be mentioned. These solvents are used alone or in combination.

The reaction temperature for this reduction is not particularly limited and the reaction is generally carried out under cooling or heating.

When the protecting group is tri-substituted silyl, deprotection can also be carried out using tetrabutylammonium fluoride, hydrogen fluoride, cesium fluoride, potassium fluoride and the like.

In this step, the steric configuration of compound (III) or a salt thereof is retained as it is in the obtained compound (IIIa) or a salt thereof.

### Step 4 (Racemization)

The racemization is carried out, for example, by the following step.

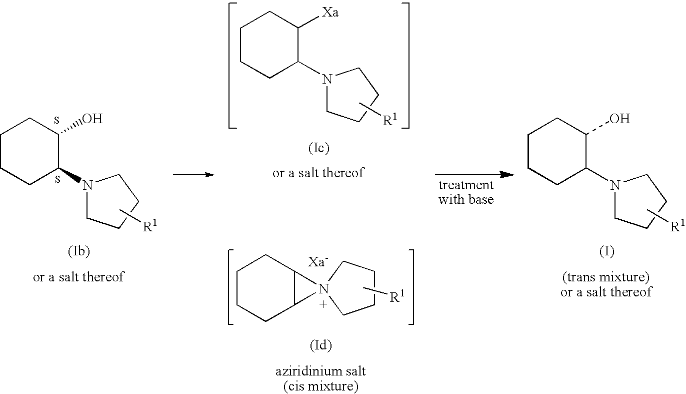

wherein each symbol is as defined above.

In this step, compound (I), which is a trans mixture, or a salt thereof can be produced by subjecting compound (Ib) (SS form) or a salt thereof, which is the diastereomer of compound (Ia) (RR form) or a salt thereof obtained by the optical resolution in Step 1, to a racemization.

As a method for racemization employed in this step, a method comprising treating compound (Ic) or a salt thereof obtained by converting the hydroxyl group of compound (Ib) to a leaving group (Xa), or compound (Id) (cis mixture) which is an aziridinium salt thereof produced during the process, or a mixture thereof, with a base, is preferable.

As the leaving group (Xa) of compound (Ic), for example, halogen atom (fluorine atom, chlorine atom, bromine atom, iodine atom), methanesulfonyloxy group, p-toluenesulfonyloxy group and the like can be mentioned. Preferred is halogen atom, and chlorine atom is particularly preferable.

A step for converting compound (Ib) or a salt thereof to compound (Ic) or a salt thereof, and/or compound (Id) can be performed according to a conventionally known method and, for example, a method described in Helv. Chem. Acta, 30, 1454, (1947) or a method analogous thereto. Preferably, the hydroxyl group of compound (Ib) or a salt thereof can be converted to a leaving group (Xa) by adding methanesulfonyl halide (preferably methanesulfonyl chloride), or p-toluenesulfonyl halide (preferably p-toluenesulfonyl chloride) to compound (Ib) or a salt thereof in the presence of a base.

As the base to be used for the racemization, inorganic bases and organic bases, such as alkali metals (e.g., sodium, potassium etc.), alkaline earth metals (e.g., magnesium, calcium etc.), hydroxide, carbonate and bicarbonate thereof, trialkylamines (e.g., trimethylamine, triethylamine etc.) can be mentioned. Of these, organic bases are preferable, trialkylamines are more preferable, and triethylamine is particularly preferable.

The amount of methanesulfonyl halide or p-toluenesulfonyl halide to be used is generally 1 to 2 mol per 1 mol of compound (Ib) or a salt thereof.

The compound (Ic) or a salt thereof, and/or compound (Id) may be isolated, but the reaction mixture after the completion of the reaction may be used for the next base treatment without isolation.

As the base for the base treatment of compound (Ic) or a salt thereof, and/or compound (Id), inorganic bases and organic bases, such as alkali metals (e.g., sodium, potassium etc.), alkaline earth metals (e.g., magnesium, calcium etc.), hydroxide, carbonate and bicarbonate thereof, trialkylamines (e.g., trimethylamine, triethylamine etc.) can be mentioned. Of these, inorganic bases are preferable, hydroxide, carbonate and bicarbonate of alkali metals (e.g., sodium, potassium etc.) are more preferable, and sodium hydrogen carbonate is particularly preferable.

The amount of the base to be used for each of the racemization and the base treatment is generally 0.5 to 100 mol relative to 1 mol of compound (Ib) or a salt thereof.

As the solvent to be used, a solvent that does not adversely affect the reaction of the racemization or the base treatment, and a solvent suitable for compound (Ib) or a salt thereof, compound (Ic) or a salt thereof, and compound (Id) can be used. For example, esters such as ethyl acetate, methyl acetate etc.; ethers such as diethyl ether, tetrahydrofuran etc.; hydrocarbons such as toluene etc.; halogenated hydrocarbons such as chloroform, dichloromethane etc.; alcohols such as methanol, ethanol, isopropyl alcohol etc.; water and the like can be mentioned. These solvents may be used alone or in combination. As preferable example, a mixture of tetrahydrofuran and water can be mentioned.

As the conditions (reaction temperature etc.) of the reaction, those suitable for the reaction are employed, and cooling to under heating is generally employed.

In Step 1-4, purification can be conducted as necessary by a known purification means such as recrystallization, column chromatography, thin layer chromatography, high performance liquid chromatography and the like. The compound can be identified by NMR spectrum analysis, mass spectrum analysis, IR spectrum analysis, elemental analysis, melting point measurement and the like.

The compound (I), compound (Ia), compound (Ib), compound (Ic), compound (III) and compound (IIIa) may be converted to a salt thereof with an acid such as inorganic acids (e.g., hydrochloric acid, hydrobromic acid, sulfuric acid, phosphoric acid and the like), organic carboxylic acids (e.g., formic acid, acetic acid, trifluoroacetic acid, maleic acid, tartaric acid and the like), sulfonic acids (e.g., methanesulfonic acid, benzenesulfonic acid, p-toluenesulfonic acid and the like), acidic amino acids (e.g., arginine, aspartic acid, glutamic acid and the like) and the like.

The compound (Ia):

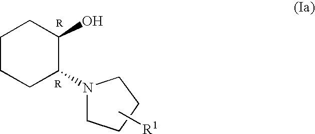

wherein R¹ is as defined above, and a salt thereof, are novel compounds, and useful intermediates for the industrially valuable production method of the present invention of compound (IIIa) having an ion channel controlling action and useful as an agent for the prophylaxis and/or treatment of various diseases such as arrhythmia (atrial arrhythmia, ventricular arrhythmia), fibrillation (atrial fibrillation, ventricular fibrillation), flutter (atrial flutter, ventricular flutter) and so on.

The production method of the present invention affords the following advantages as compared to conventional methods of WO2004/099137 and the like.

Optical resolution of compound (I), a trans mixture, by crystallization (preferably fractional crystallization by salt formation) affords optically active compound (Ia) with high purity and yield. Since chiral columns such as HPLC and the like used in conventional methods are not used, optical resolution can be performed efficiently.

Furthermore, conventional methods require optical resolution after reaction with relatively expensive DMPE (compound (5R)), which in turn increases the cost. In the present invention, since optical resolution is performed before reaction with a reactive derivative (compound (II)) of a compound such as DMPE and the like, which is advantageous in terms of cost.

In the below-mentioned Examples, where compound (7) (compound A) is obtained from compound (1), the yield is about 10% by conventional methods but 18-23% by the method of the present invention, that is, compound A was obtained in an about twice higher yield in the method of present invention. Therefore, the present invention affords the object compound (IIIa) from compound (I) in a higher yield, and is highly advantageous in cost.

In conventional methods, about half of compound (4R) obtained through 4 steps can not be used as a starting material of compound A. In the present invention, however, an other optically active compound (compound (Ib)) which is the diastereomer of compound (Ia) obtained by optical resolution can be converted to a trans mixture (compound (I)), which is a compound before optical resolution, by racemization in one step. Therefore, compound (I) can be used for the optical resolution and the present invention is still more advantageous in cost.

Moreover, in conventional methods, compound (4R) is reacted with methanesulfonyl chloride to convert OH group to chloro group and then reacted with DMPE. However, the present inventors have found that, when optically active compound (Ia) is reacted with methanesulfonyl chloride as in conventional methods, the steric configuration thereof cannot be retained. In the present invention, therefore, compound (II) obtained by converting OH group of a compound such as DMPE and the like to a highly reactive leaving group is reacted with compound (Ia) and then the resulting compound (compound (III)) is deprotected, whereby the steric configuration of compound (Ia) is retained through the object compound (IIIa).

Therefore, the production method of the present invention enables efficient and stable supply in a high yield at a lower cost, and is an industrially highly advantageous method, which can provide an optically active cyclohexane ether compound extremely useful as a pharmaceutical agent.

### EXAMPLES

The present invention is explained in detail in the following by referring to Examples, which are not to be construed as limitative.

### Example 1

### Synthesis of (1R,2R)-2-[(3R)-3-benzyloxy-1-pyrrolidinyl]cyclohexanol ½ di-p-toluoyl-L-tartarate (compound (2))

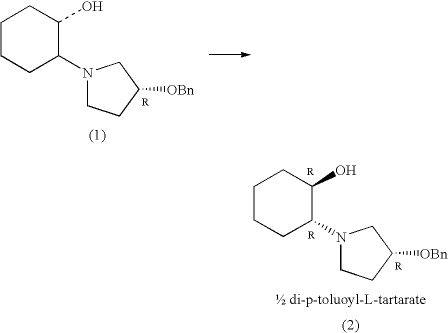

wherein Bn is a benzyl group.

2-[(3R)-3-Benzyloxy-1-pyrrolidinyl]cyclohexanol (compound (1), trans mixture) (112.0 g, purity 81.1%) was added to ethyl acetate (2240 ml), and the mixture was dissolved at 25-28° C. Thereto was added di-p-toluoyl-L-tartaric acid (78.6 g) and the mixture was stirred at the same temperature for about 5 hr (crystals precipitate on the way), cooled to 0-10° C. and stirred overnight at the same temperature. The precipitated crystals were collected by filtration and washed with ethyl acetate (336 ml) cooled in advance. The obtained crystals were vacuum dried to give crude crystals of (1R,2R)-2-[(3R)-3-benzyloxy-1-pyrrolidinyl]cyclohexanol ½ di-p-toluoyl-L-tartarate (80.4 g, de 90%). The crude crystals (80.0 g) and water (70 ml) were added to isopropyl alcohol (352 ml) and the mixture was heated to about 50° C. to dissolve the mixture. After confirmation of dissolution, the mixture was cooled to about 30° C. After confirmation of crystal precipitation, the mixture was stirred at the same temperature for about 1.5 hr, cooled to 0-10° C. and stirred at about 3 hr. The crystals were collected by filtration and washed with ethyl acetate (288 ml) cooled in advance. The obtained crystals were vacuum dried to give (1R,2R)-2-[(3R)-3-benzyloxy-1-pyrrolidinyl]cyclohexanol ½ di-p-toluoyl-L-tartarate (compound (2)) (64.3 g, de 100%, yield based on purity 41.0%).

### Compound (2):

MS: m/z 276 (M−½ di-p-toluoyl-L-tartaric acid+H)^{+ 1}H NMR (400 MHz, DMSO-d₆): 67 1.18-1.29 (4H, m), 1.59-1.65 (2H, m), 1.85 (3H, m), 2.00 (1H, m), 2.34 (3H, s), 2.59-3.49 (6H, m), 4.13 (1H, m), 4.44 (2H, s), 5.58 (1H, s), 7.27 (2H, d, J=8.2 Hz), 7.29-7.37 (5H, m), 7.79 (2H, d, J=8.2 Hz)

### Example 2

### Synthesis of (3R)-3-benzyloxy-1-{(1R,2R)-2-[2-(3,4-dimethoxyphenyl)ethoxy]cyclohexyl}pyrrolidine (compound (5))

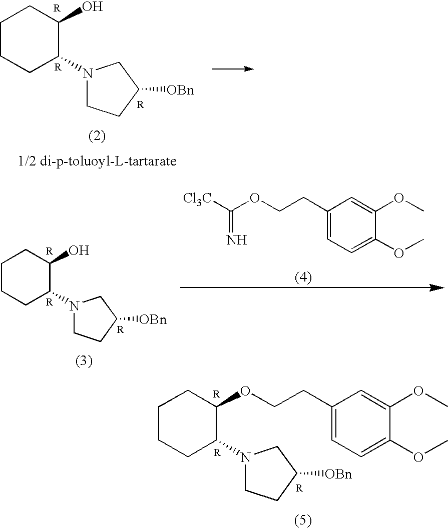

wherein Bn is as defined above.

(1R,2R)-2-[(3R)-3-Benzyloxy-1-pyrrolidinyl]cyclohexanol ½ di-p-toluoyl-L-tartarate (compound (2)) (20.0 g, purity 100%) was added to a mixed solution of toluene (400 ml) and water (100 ml) and the mixture was suspended at 26° C. 24% Aqueous sodium hydroxide solution (7.8 g) was added dropwise to dissolve crystals. After confirmation of dissolution, the mixture was partitioned and the organic layer was washed twice with water (200 ml). The organic layer was concentrated to 200 ml under reduced pressure to give a solution of (1R,2R)-2-[(3R)-3-benzyloxy-1-pyrrolidinyl]cyclohexanol (compound (3)).

3,4-Dimethoxyphenethyl-N-trichloroacetimidate (compound (4)) (27.9 g) was added to the above-mentioned solution, and the mixture was cooled to about 5° C. and trifluoromethanesulfonic acid (10.3 g) was added dropwise at not more than 25° C. After completion of the dropwise addition, the mixture was reacted at 24-25° C. for about 6 hr. After the completion of the reaction, isopropyl alcohol (40 ml) was added and the mixture was stirred for about 30 min. Thereafter, water (100 ml) was added and the mixture was partitioned. The organic layer was washed twice with 8% aqueous sodium hydrogencarbonate (100 ml). The organic layer was concentrated under reduced pressure to give an oil. This oil was purified by alumina column chromatography (Al₂O₃ 400 g, n-hexane→n-hexane:ethyl acetate=50:1). A solution of the fraction was concentrated to 400 ml, and water (200 ml) was added and adjusted to pH 0.5-1.0 with 6N hydrochloric acid. The aqueous layer was separated, washed twice with n-hexane (40 ml) and washed with methyl-t-butyl ether (40 ml). The aqueous layer was adjusted to pH 10-12 with 24% aqueous sodium hydroxide solution. The mixture was extracted with ethyl acetate (400 ml) and the obtained organic layer was concentrated to dryness to give (3R)-3-benzyloxy-1-{(1R,2R)-2-[2-(3,4-dimethoxyphenyl)ethoxy]cyclohexyl}pyrrolidine (compound (5)) (12.8 g, purity 95.6%, yield based on purity 65.1%) as an oil.

### Compound (5):

MS: m/z 440 (M+H)^{+ 1}H NMR (200 MHz, CDCl₃): δ 1.17-2.02 (10H, m), 2.22 (1H, m), 2.45-2.78 (6H, m), 3.33-3.67 (3H, m), 3.69 (3H, s), 3.71 (3H, s), 4.00 (1H, m), 4.40 (2H, s), 6.72 (1H, dd, J=8.1, 1.8 Hz), 6.82 (1H, d, J=8.1 Hz), 6.84 (1H, s), 7.23-7.37 (5H, m)

### Example 3

### Synthesis of (3R)-1-{(1R,2R)-2-[2-(3,4-dimethoxyphenyl)ethoxy]cyclohexyl}pyrrolidinol hydrochloride (compound (7))

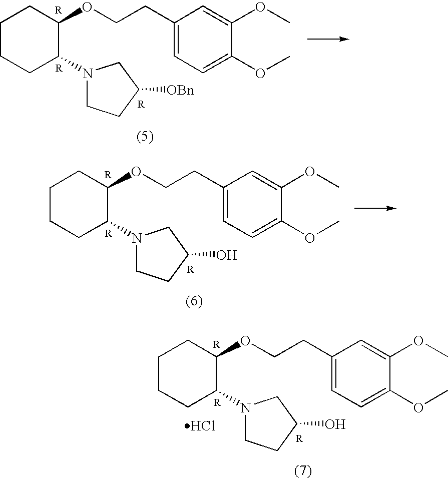

wherein Bn is as defined above.

(3R)-3-Benzyloxy-1-{(1R,2R)-2-[2-(3,4-dimethoxyphenyl)ethoxy]cyclohexyl}pyrrolidine (compound (5)) (12.7 g, purity 95.6%) was dissolved in methanol (64 ml) and formic acid (64 ml) was added under cooling. Then, 10% palladium carbon (12.7 g) was added to allow reaction at 35-40° C. for about 3 hr. After the completion of the reaction, palladium carbon was filtered off. Water (20 ml) was added to the filtrate and methanol was evaporated under reduced pressure. Water (20 ml) was added and the mixture was adjusted to 10-12 with 24% aqueous sodium hydroxide solution under cooling. The mixture was extracted three times with isopropyl acetate (64 ml) and the organic layer was washed with water (64 ml). The organic layer was dried over magnesium sulfate and concentrated to dryness to give (3R)-1-{(1R,2R)-2-[2-(3,4-dimethoxyphenyl)ethoxy]cyclohexyl}pyrrolidinol (compound (6)) as an oil (8.8 g). The obtained oil was in dissolved in isopropyl alcohol (IPA, 88 ml) and the mixture was cooled to 0-10° C. 2N Hydrochloric acid/IPA (16.4 ml) was added dropwise over about 15 min while maintaining the same temperature and then methyl-t-butyl ether (MTBE, 88 ml) was added dropwise over about 30 min. After stirring for aging at the same temperature for about 4 hr, the precipitated crystals were collected by filtration and washed with MTBE/IPA (1:1) mixture (35 ml) cooled in advance. The obtained crystals were vacuum dried to give (3R)-1-{(1R,2R)-2-[2-(3,4-dimethoxyphenyl)ethoxy]cyclohexyl}pyrrolidinol hydrochloride (compound (7)) (7.5 g, purity 99.1%, yield based on purity 69.8%).

### Compound (7):

MS: m/z 350 (M−HCl+H)^{+ 1}H NMR (400 MHz, DMSO-d₆): δ 1.03-1.23 (3H, m), 1.38 (1H, m), 1.66-1.81 (3H, m), 1.91-2.08 (2H, m), 2.23 (1H, m), 2.78 (2H, m), 3.08-3.60 (7H, m), 3.71 (3H, s), 3.74 (3H, s), 3.78 (1H, m), 4.29 (1H, s), 5.43 (1H, brd), 6.76 (1H, d, J=8.0 Hz), 6.86 (1H, d, J=8.0Hz), 6.87 (1H, s)

Additional tests were performed according to the same methods as in Examples 1 to 3. To compare the yield, compound A was synthesized from 4R according to the method described in WO2004/099137 (Scheme 1), and the yield based on purity was calculated. The results are shown in the following.

TABLE 1

| | from compound | from compound | from compound |
|---|---|---|---|
| | (1) to | (5) to compound | (1) to |
| | compound (5) | (7) | compound (7) |
| 1st time | 26.7% | 69.8% | 18.6% |
| 2nd time | 27.8% | 84.6% | 23.6% |
| WO2004/ | 13.2% | 75.7% | 10.0% |
| 099137 | | | |

### Example 4

### Synthesis of 2-[(3R)-3-benzyloxy-1-pyrrolidinyl]cyclohexanol (compound (1), trans mixture) by racemization of (1S,2S)-2-[(3R)-3-benzyloxy-1-pyrrolidinyl]cyclohexanol (compound (8)) and synthesis of (1R,2R)-2-[(3R)-3-benzyloxy-1-pyrrolidinyl]cyclohexanol-½ di-p-toluoyl-L-tartarate (compound (2)) using the obtained compound (1)

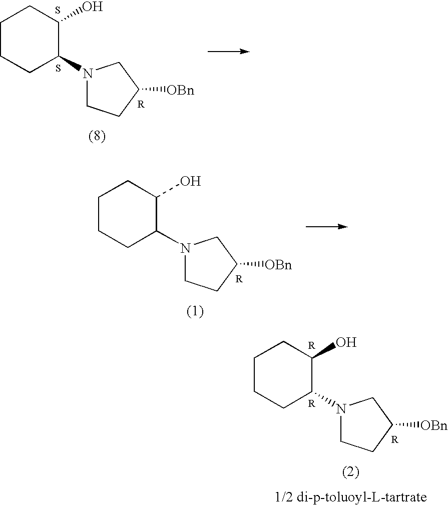

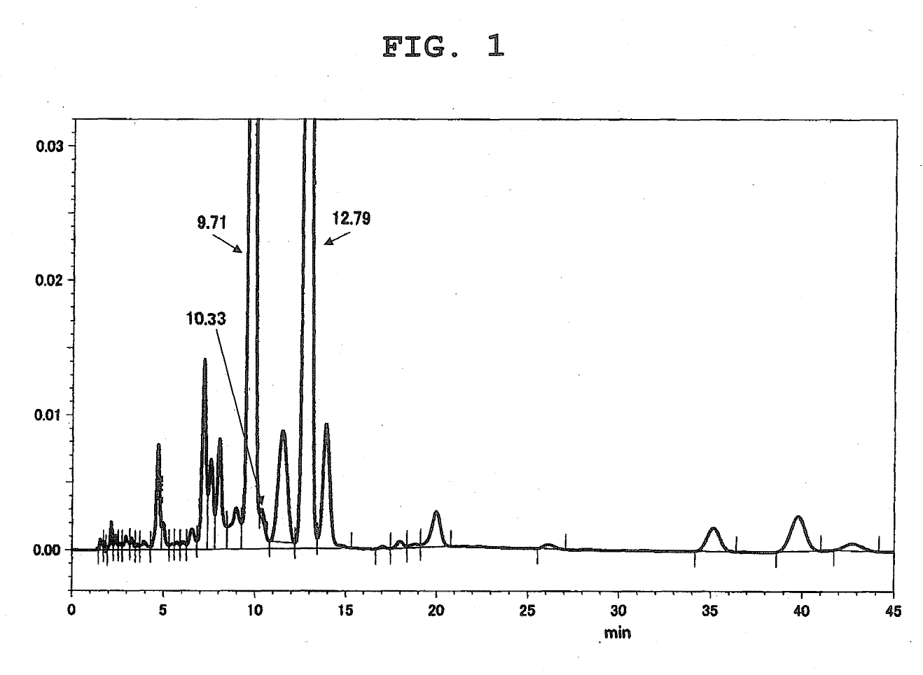

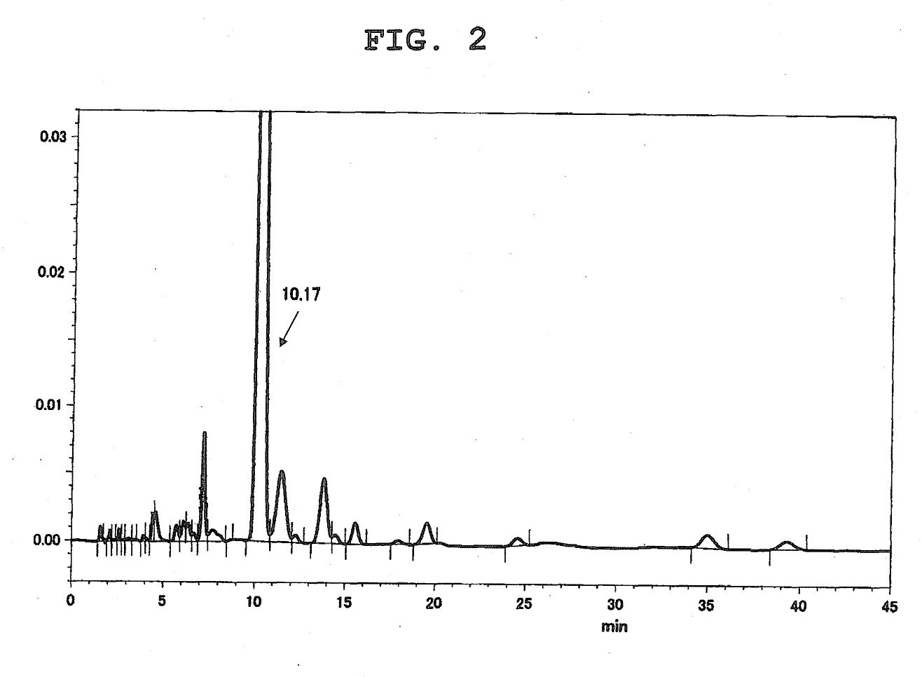

After crude crystals of (1R,2R)-2-[(3R)-3-benzyloxy-1-pyrrolidinyl]cyclohexanol-½ di-p-toluoyl-L-tartrate were collected by filtration in Example 1, the obtained filtrate (100 mL) was washed with 1N aqueous sodium hydroxide solution (20 mL) and tap water (twice with 20 mL). The organic layer was concentrated under reduced pressure. Toluene (20 mL) was added to the residue and the mixture was concentrated, and these operations were repeated. The residue was dissolved in tetrahydrofuran (15 mL) and triethylamine (1.50 g) and methanesulfonyl chloride (1.45 g) was added dropwise at 0-15° C. The mixture was stirred at the same temperature for one day. At this time, the presence of the aziridinium salt and 1-chloro compound in the reaction mixture was confirmed by HPLC (HPLC conditions are shown below). The HPLC chart is shown in FIG. 1. Then, 10% aqueous sodium hydrogen carbonate solution (15 mL) was added to the reaction mixture and the mixture was stirred at 60-65° C. for 3 days. At this time, the disappearance of the aziridinium salt and 1-chloro compound from the reaction mixture was confirmed by HPLC (HPLC conditions are shown below). The HPLC chart is shown in FIG. 2. Then, the reaction mixture was extracted with toluene, and the organic layer was concentrated under reduced pressure to give compound (1) (trans mixture, 2.87 g) as an oil.

The obtained oil was dissolved in ethyl acetate (58 mL), di-p-toluoyl-L-tartaric acid (1.12 g) was added, and the mixture was stirred at 25-30° C. for 4 hrs. The mixture was cooled to 0-10° C., and the precipitate was collected by filtration and dried. The obtained crude crystal (2.24 g) was recrystallized from aqueous isopropanol solution to give (1R,2R)-2-[(3R)-3-benzyloxy-1-pyrrolidinyl]cyclohexanol.½ di-p-toluoyl-L-tartarate (1.85 g, de 100%).

### HPLC Measurement Conditions

packed column: Inertsil® ODS-3 (particle size 5 μm, inner diameter 4.6 mm, length 250 mm)

mobile phase: A) perchloric acid buffer* (pH=2.5), B) IPA (* NaClO₄.H₂O (14.05 g) was dissolved in distilled water (1000 mL) and adjusted to pH=2.5 with perchloric acid (1→20))

| Time (min) | SOLUTION A | SOLUTION B | elution |
|---|---|---|---|
| 0 | 85 | 15 | equilibrated |
| 0-20 | 85 | 15 | isocratic |
| 20-35 | 85 → 50 | 15 → 50 | gradient |
| 35-70 | 50 | 50 | isocratic |
| 70-85 | 85 | 15 | Re-equilibrated |

flow rate : 0.9 mL/min,

wavelength : 220 nm

column temperature: 50° C.

injection volume: 5 μL

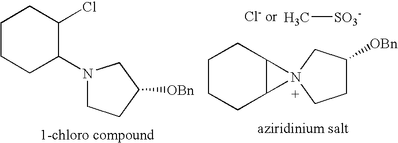

### INDUSTRIAL APPLICABILITY

The production method of the present invention enables efficient and stable supply of an optically active cyclohexane ether compound extremely useful as a pharmaceutical agent in a high yield at a lower cost. And, an intermediate useful for the production method of the present invention can be provided.

This application is based on application No. 60/642,998 filed in the United States of America, the contents of which are incorporated hereinto by reference.

1. A production method of an optically active compound represented by the formula (IIIa): 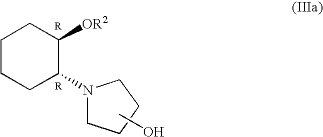
wherein R² is an optionally substituted aryl lower alkyl group, or a salt thereof, which comprises subjecting a compound represented by the formula (I), which is a trans mixture: 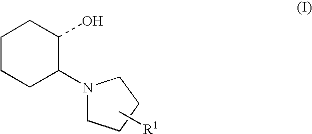
wherein R¹ is an optionally protected hydroxy group, or a salt thereof, to optical resolution using ethyl acetate as a solvent to give an optically active compound represented by the formula (Ia): 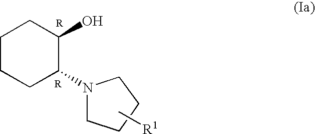
wherein R¹ is as defined above, or a salt thereof, reacting the resulting compound or a salt thereof with a compound represented by the formula (II): X—R² wherein R² is as defined above and X is a leaving group, using a catalytic amount of trifluoromethanesulfonic acid as a Lewis acid, to give an optically active compound represented by the formula (III): 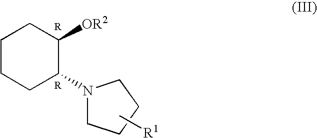
wherein R¹ and R² are as defined above, or a salt thereof, and then eliminating, when R¹ is a protected hydroxy group, the hydroxyl-protecting group from the resulting compound or a salt thereof.

2. A production method of an optically active compound represented by the formula (III): 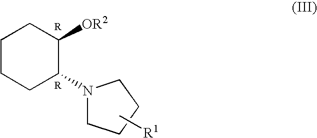
wherein R¹ is an optionally protected hydroxy group, and R² is an optionally substituted aryl lower alkyl group, or a salt thereof, which comprises subjecting a compound represented by the formula (I), which is a trans mixture: 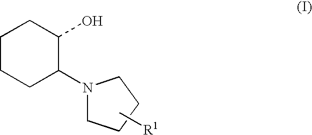
wherein R¹ is as defined above, or a salt thereof, to optical resolution using ethyl acetate as a solvent to give an optically active compound represented by the formula (Ia): 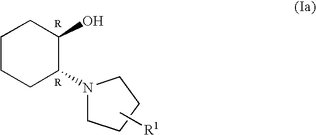
wherein R¹ is as defined above, or a salt thereof, and then reacting the resulting compound or a salt thereof with a compound represented by the formula (II): X—R² wherein R² is as defined above and X is a leaving group.

3. A production method of an optically active compound represented by the formula (Ia): 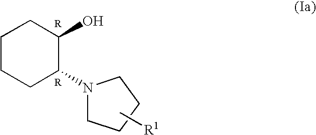
wherein R¹ is an optionally protected hydroxy group, or a salt thereof, which comprises subjecting a compound represented by the formula (I), which is a trans mixture: 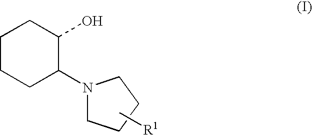
wherein R¹ is as defined above, or a salt thereof, to optical resolution using ethyl acetate as a solvent.

4. The production method of any of claims 1 to 3, wherein the optical resolution is preformed by fractional crystallization by salt formation.

5. A production method of an optically active compound represented by the formula (IIIa): 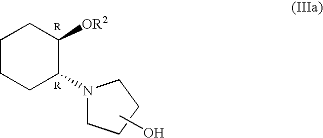
wherein R¹ is an optionally substituted aryl lower alkyl group, or a salt thereof, which comprises reacting an optically active compound represented by the formula (Ia): 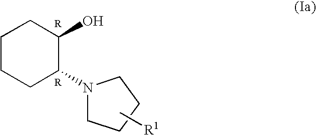
wherein R¹ is an optionally protected hydroxy group, or a salt thereof, with a compound represented by the formula (II): X—R² wherein R² is as defined above and X is a leaving group, using a catalytic amount of trifluoromethanesulfonic acid as a Lewis acid, to give an optically active compound represented by the formula (III): 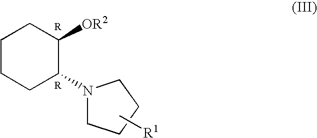
wherein R¹ and R² are as defined above, or a salt thereof, and then eliminating, when R¹ is a protected hydroxy group, the hydroxyl-protecting group from the resulting compound or a salt thereof.

6. A production method of an optically active compound represented by the formula (III): 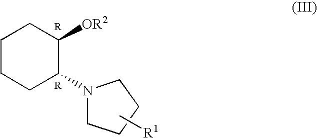
wherein R¹ is an optionally protected hydroxy group, and R² is an optionally substituted aryl lower alkyl group, or a salt thereof, which comprises reacting an optically active compound represented by the formula (Ia): 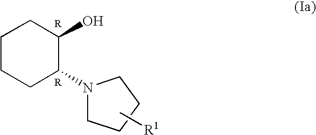
wherein R¹ is as defined above, or a salt thereof, with a compound represented by the formula (II): X—R² wherein R² is as defined above and X is a leaving group using a catalytic amount of trifluoromethanesulfonic acid as a Lewis acid.

7. A production method of a compound represented by the formula (I), which is a trans mixture: 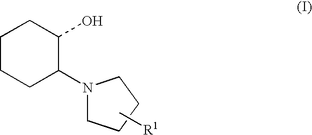
wherein R¹ is an optionally protected hydroxy group, or a salt thereof, which comprises subjecting an optically active compound represented by the formula (Ib): 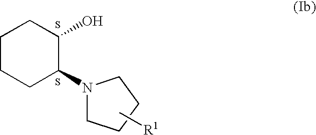
wherein R¹ is as defined above, or a salt thereof, to a racemization.

8. The production method of any of claims 1 to 3, wherein the compound represented by the formula (I), which is a trans mixture: 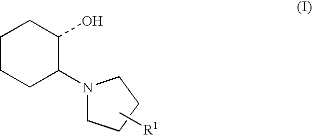
wherein R¹ is an optionally protected hydroxy group, or a salt thereof, is obtained by subjecting an optically active compound represented by the formula (Ib): 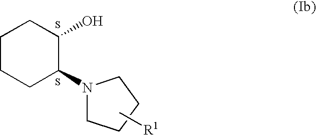
wherein R¹ is as defined above, or a salt thereof, to a racemization.

9. (canceled)
